(19) United States
(12) Patent Application Publication
(10) Pub. No.: US 2022/0218326 A1
(43) Pub. Date: Jul. 14, 2022

(54) EXPANDABLE RETRACTOR BLADE ASSEMBLY

(71) Applicant: ROB DICKERMAN, PLANO, TX (US)

(72) Inventor: ROB DICKERMAN, PLANO, TX (US)

(21) Appl. No.: 17/145,081

(22) Filed: Jan. 8, 2021

(51) Int. Cl. A61B 17/02

(52) U.S. Cl. CPC A61B 17/0206; A61B 2017/0256

(57) ABSTRACT

An expandable blade assembly for use with a medical retractor includes a first blade, a first pivoting blade, and a second pivoting blade. The first pivoting blade pivots about an axis and the second pivoting blade pivots about an axis. The points of rotation (i.e. pivoting) of the pivoting blades and are at different locations on the first blade. Each pivoting blade is coupled to blade 103 and permitted to rotate about its respective axis. The rotation of each pivoting blade may occur independent of the other or as a collective group of pivoting blades. Rotation of the pivoting blades may be done either in opposing directions from the other pivoting blades or in the same direction. A gear may be used to permit selective rotation of the pivoting blades.

### BACKGROUND OF THE INVENTION

### 1. Field of the Invention

The present application relates to a medical device, and more particularly to a medical device inserted into the body operable in relation to the spine and neighboring tissue.

### 2. Description of Related Art

Retractors are commonly used medical devices, or surgical instruments, which are used to separate the edges of a surgical incision or wound so as to hold back underlying organs and tissue. This is necessary to permit access to body parts under the skin layer. These are typically simple handheld tools that possess a curved or angled blade and are fitted with a handle. Often the blades are located on opposing arms and are separated by a spring or gear. The arms are used to direct the location of the blade. Each blade is a singular member having a set width and length. When in place, the static blades apply pressure within the body to hold back tissue and other organs.

A disadvantage of conventional retractors and the corresponding blades is that it is desirable to maintain as minimal of an incision as possible. Blades are generally fixed in width and thickness therefore, the size of the incision and the size of the blade correspond to one another. When within the body it is often desired to have a blade hold back more area of tissue and organs. However, this is not possible unless the incision is increased to facilitate a larger blade. This has obvious disadvantages.

Although strides have been made in surgical procedures, shortcomings remain. It is desired that an assembly be provided that allows for the size of the blades to be selectively expanded and retracted within the body so as to maintain minimal incision sizes.

### BRIEF SUMMARY OF THE INVENTION

It is an object of the present application to provide an expandable blade for use with retractors in the medical and surgical field. The expandable blade assembly is configured to selectively increase and decrease in width inside the body. A medical professional is able to operate the blade assembly from outside the body. The adjustable width of the blades permit incisions to remain and minimal as possible while maximizing the amount of tissue that can be displaced within the body.

It is a further object of the present application that a plurality of blades are aligned in a stacked configuration such that they are oriented in an overlapping manner when closed. When opened, the blades fan out radially in a relatively planar manner. This would allow each arm of a retractor to operate a blade assembly that is expandable in width. One or more contoured areas, such as bends, in the assembly may be used to enhance visibility by more readily holding back internal tissue.

Additionally, the blade assembly is configured to be coupled to one or more types of retractors. A gear is used to operate the blades wherein the blades are configured to open and close as a gear is rotated. The blades themselves optionally include gear teeth to engage the gear so as to facilitate operation between the closed and open positions. The particular design of the expandable blade assembly is equally suitable for applications outside the medical industry and may be used in such manners as deemed useful.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective view of a front view of an expandable blade assembly according to an embodiment of the present application, aligned in a closed position.

FIG. 2 is a front view of the expandable blade assembly of FIG. 1 aligned in an open position.

FIG. 3 is a perspective view of an alternate embodiment of the expandable blade assembly of FIGS. 1 and 2.

FIG. 4 is a side view of the expandable blade assembly of FIG. 3.

FIG. 5 is an alternate perspective view of the expandable blade assembly of FIG. 3 without an attachment member and keyed tool.

FIG. 6 is a front view of the expandable blade assembly of FIG. 5 in a closed position.

FIG. 7 is a front view of the expandable blade assembly of FIG. 6 in an open position.

FIG. 8 is a rear view of the expandable blade assembly of FIG. 7.

FIG. 9 is a top view of the expandable blade assembly of FIG. 5.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method in accordance with the present application overcomes one or more of the above-discussed problems commonly associated with the prior art discussed previously. In particular, the expandable blade assembly of the present application is configured to operate between an open and closed position wherein a plurality of blades are selectively overlapped so as to minimize an overall width. In operation the blades, either simultaneously or individually, may be rotated about an axis to increase the collective width of the plurality of blades when opened. One or more blades may be operated. The movement of each blade may take place along a single plane or in a nonplanar displacement. These and other unique features are discussed below and illustrated in the accompanying drawings.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

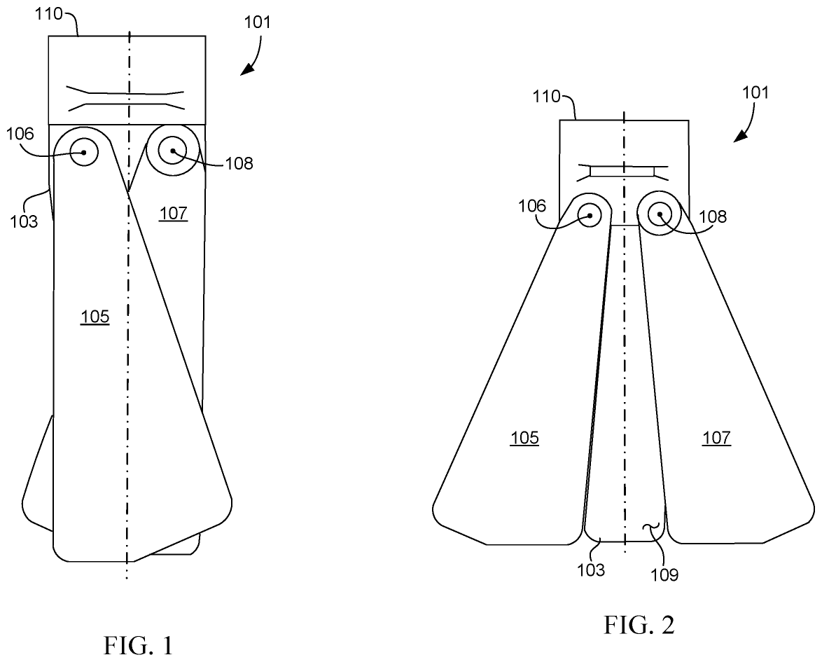

Referring now to FIGS. 1 and 2 in the drawings, an expandable blade assembly is illustrated. Expandable blade assembly 101 is configured to permit the selective widening and narrowing of the collective width of a plurality of blades which are selectively nestled together in an overlapping manner. Assembly 101 is configured to operate in a closed position and an open position. When in the closed position, a series of blades are overlapped as seen in the front view of FIG. 1. The collective width is minimized. When in the open position, a series of blades are pivoted relative to each other so as to expand the collective width as seen in FIG. 2.

Assembly 101 is configured to nestle together in a closed position and pass through an incision in a body. The minimal width of the closed position allows the incision to remain as minimal as possible. Once inserted, the pivoting blades of assembly 101 may be pivoted into an open position. In this position, the collective width is increased. This benefits a medical professional as a singular blade/blade assembly can expand in width greater than the length of the incision so as to displace or move more tissue of the body compared to a static blade only. Greater visibility is provided to the medical professional all while maintaining a narrower shorter incision.

As seen in FIGS. 1 and 2, assembly 101 includes a first blade 103, a first pivoting blade 105 and a second pivoting blade 107. The first pivoting blade 105 pivots about an axis 106 and the second pivoting blade 107 pivots about an axis 108. The points of rotation (i.e. pivoting) of blades 105 and 107 are at different locations. Each blade 105/107 is coupled to blade 103 and permitted to rotate about its respective axis. The rotation of each blade may occur independent of the other or as a collective group of blades 105/107. Rotation of blades 105/107 may be done either in opposing directions from the other pivoting blades or in the same direction.

To facilitate the overlapping orientation of blades 105/107, each blade 105/107 is offset from a front surface 109 of blade 103. Either blade 105/107 may be located adjacent to or closest to front surface 109. It is important that the blades 105/107 overlap at some point.

It is understood that the width of blade 103 may be set and any width. In some embodiments blades 105/107 do not have to overlap. For example, each blade 105/107 may be equal to or less than half the width of blade 103, such that in a closed position no overlapping occurs and the collective width is no larger than the width of the first blade 103. In such an embodiment, the offset nature of blades 105/107 are not needed. Naturally this decreases the overall achievable width of assembly 101, however, it is conceivable for operational purposes herein. An advantage of overlapping permits a maximum width when in the open position. It is understood that FIG. 1 illustrates blades 105/107 as extending outward beyond the width of blade 103. Blade shape has a bearing on overall width in both closed and open positions and the blade shape herein is not meant to be limiting, but merely exemplary in nature.

Blades 105/107 and first blade 103 are typically relatively thin pieces of material that are hardened into a particular shape. The width of each is considerably larger than the thickness. Blades 105/107 pivot along a set plane. As noted above, blades 105/107 may share the same plane or may be pivotal along different planes of rotation. Other embodiments may permit nonplanar rotations.

In particular with FIGS. 1 and 2 specifically, assembly 101 includes first blade 103 with blades 105/107 coupled thereto in a manner permitting pivoting about different axis. The pivoting blades operate between the closed and open positions so as to collectively increase the width of the entire assembly. Each blade is selectively offset from front surface 109 a set distance. Assembly 101 is coupled to a retractor in operation along an exterior surface, for example upper surface 110 could be feasible.

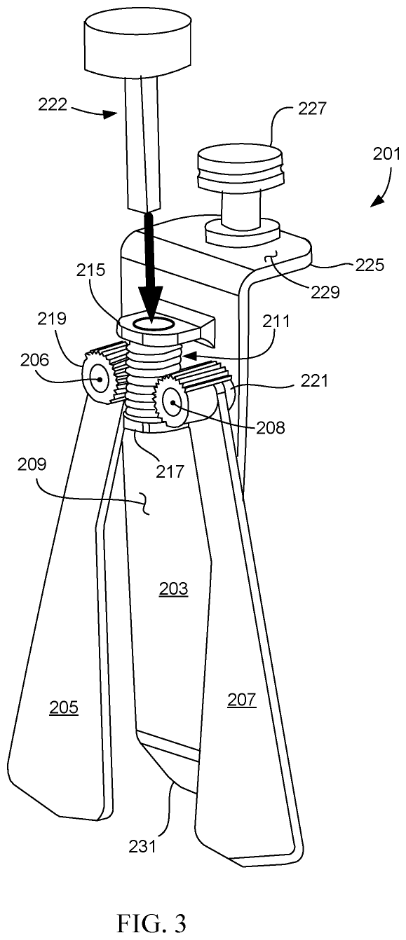

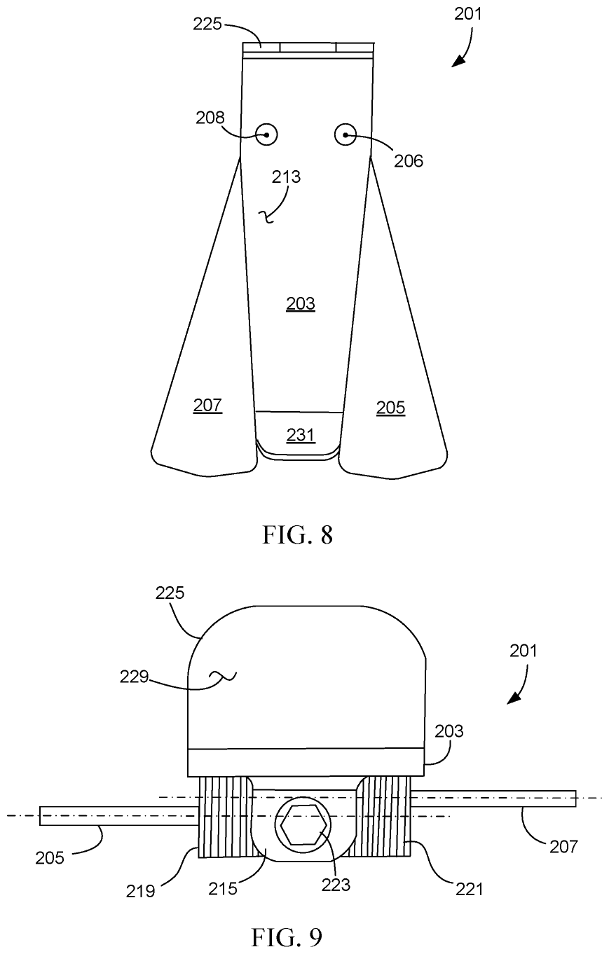

Referring now also to FIGS. 3-9 in the drawings, views of an expandable blade assembly 201 is illustrated. Assembly 201 is similar in form and function to that of assembly 101 except as herein identified. The functional operation is similar with respect to the pivoting blades and first blade. Similar numbering will be maintained with respect to Assembly 201 as seen with assembly 101 so as to assist in identifying similar components in form and function.

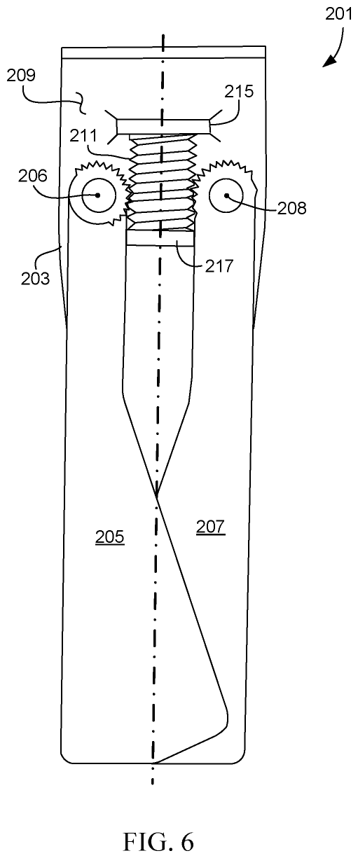

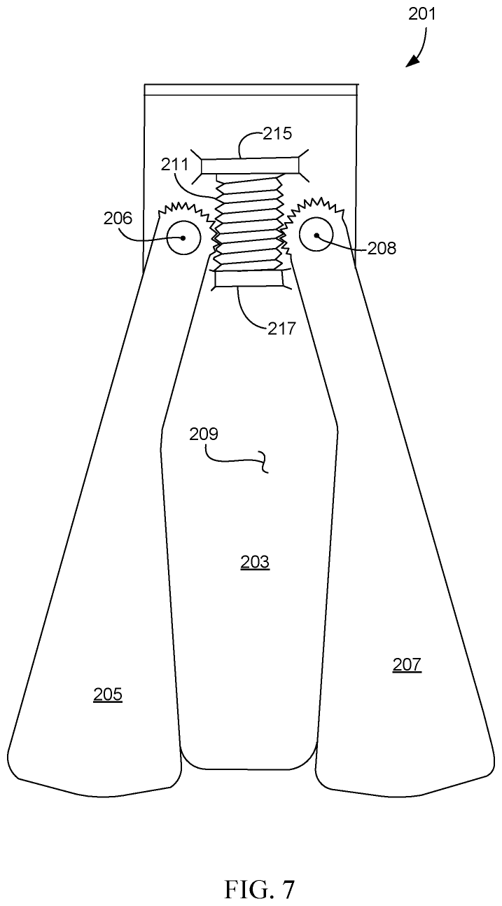

Expandable blade assembly 201 is configured to permit the selective widening and narrowing of a collective width of a plurality of blades which are selectively nestled together in an overlapping manner. Assembly 201 is configured to operate between a closed position and an open position. When in the closed position (see FIG. 6), a series of pivoting blades are oriented in front of a first blade in a manner that allows the series of blades as a whole to have a collective width a smaller than when in an open or partially open position (see FIG. 7). The collective width therefore is minimized. The operation and function is similar to that of assembly 101 noted above.

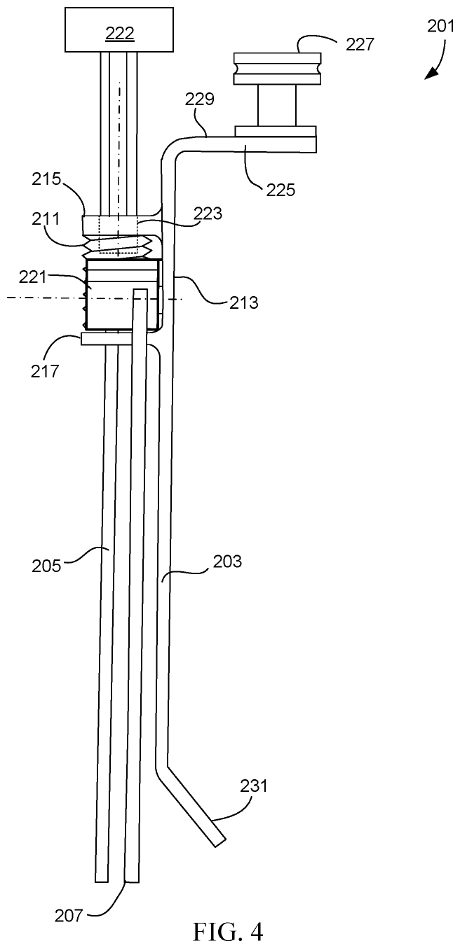

Assembly 201 includes a first blade 203, a first pivoting blade 205, a second pivoting blade 207, and a gear 211. First blade 203 includes a front surface 209 and a rear surface 213 (see FIG. 4). Each of blades 205/207 are coupled to first blade 203, such as at front surface 209. Gear 211 is coupled to front surface 209 and in threaded communication with blades 205/207 so as to facilitate operation between the open position and the closed position. As gear 211 is activated, blades 205/207 are selectively pivoted so as to influence the collective width of assembly 201.

Referring now in particular to FIGS. 6 and 7 in the drawings. Assembly 201 is configured to nestle together in a closed position (FIG. 6) and pass through an incision in a body. The minimal width of the closed position allows the incision to remain as minimal as possible. Once inserted, the pivoting blades of assembly 201 may be pivoted into an open position (FIG. 7). In this position, the collective width is increased. This benefits a medical professional as a singular blade/blade assembly can expand in width greater than the length of the incision so as to displace or move more tissue of the body compared to a static blade only. Greater visibility is provided to the medical professional all while maintaining a narrower shorter incision.

The first pivoting blade 205 pivots about an axis 206 and the second pivoting blade 207 pivots about an axis 208. The points of rotation (i.e. pivoting) of blades 205 and 207 are at different locations on blade 203. Each blade 205/207 is coupled to blade 203 and permitted to rotate about its respective axis. The rotation of each blade may occur independent of the other or as a collective group of blades 205/207. Rotation of blades 205/207 may be done either in opposing directions from the other pivoting blades or in the same direction.

To facilitate the overlapping orientation of blades 205/207, each blade 205/207 is offset from a front surface 209 of blade 203. Either blade 205/207 may be located adjacent to, or closest to, front surface 109. It is important that the blades 205/207 overlap at some point in this embodiment.

In some embodiments blades 205/207 do not have to overlap. For example, each blade 205/207 may be equal to or less than half the width of blade 103, such that in a closed position no overlapping occurs, and the collective width is no larger than the width of the first blade 203. In such an embodiment, the offset nature of blades 205/207 are not needed. Naturally this decreases the overall achievable width of assembly 201, however, it is conceivable for operational purposes herein. An advantage of overlapping permits a maximum width when in the open position. It is understood that FIG. 1 illustrates blades 205/207 being confined within the width of blade 203 in an overlapping configuration. It is understood that blade shape has a bearing on overall width in both closed and open positions and the blade shape herein is not meant to be limiting, but merely exemplary in nature. The width of any of blades 205/207 may be larger, smaller, or the same as the width of blade 203.

Blades 205/207 and first blade 203 are typically relatively thin pieces of material that are hardened into a particular shape. The width of each is considerably larger than the thickness. Blades 205/207 pivot along a set plane. As noted above, blades 205/207 may share the same plane or may be pivotal along different planes of rotation. Other embodiments may permit nonplanar rotations.

Gear 211 is coupled to front surface 209. In operation, rotation of gear 211 induces movement of blades 205/207 between positions. Gear 211 may be aligned in various ways and orientations. As depicted in the Figures, Gear 211 is aligned vertically wherein gear 211 rotates about an axis that is parallel to front surface 209. An upper mount 215 and a lower mount 217 are used to secure gear 211 in place. Each mount is also secured to blade 203 at surface 209. Although a single gear 211 is shown, it is understood that one or more gears may be used and aligned in different orientations to permit movement of blades 205/207 (i.e. linear or radial movements).

Blades 205/207 are configured similarly and are shown to have mirrored shape in the Figures. It is understood that blades 205/207 may be of similar shape or different shapes as needed. Blades 205/207 may be detachable from first blade 203 in some embodiments to permit a user to select a size and blade shape useful for a desired procedure. Blades 205/207 have a first end and a second end. The first end is configured to have an engagement member 219/221. Each engagement member is pivotally coupled to blade 203 and rotates about axis 206/208 respectively. Each engagement member also includes one or more surface treatments to selectively engage with gear 211. The surface treatments may be slots, grooves, or a series of teeth. The surface treatments are along an outer surface of the engagement members. As depicted, engagement members 219/221 are cylindrical in nature and are also partially smooth on the outer surface to facilitate attachment for the blades 205/207. In operation, as gear 211 rotates, the surface treatments of the engagement members rotated counter-clockwise or clockwise about its respective axis.

Assembly 201 also includes an activator tool 222 configured to seat within a portion of gear 211 to facilitate activation or rotation of gear 211. This is seen clearly in FIGS. 3, 5, and 9. An aperture is formed in mount 215 to permit access to a keyed slot 223 in gear 211. Tool 222 includes a matching keyed protrusion to match that of slot 223. Tool 222 may be selectively removed at any time. Gear 211 may be configured to latch at select rotational positions so as to maintain the open position of blades 205/207, wherein tool 222 may be removed while assembly 201 is in use.

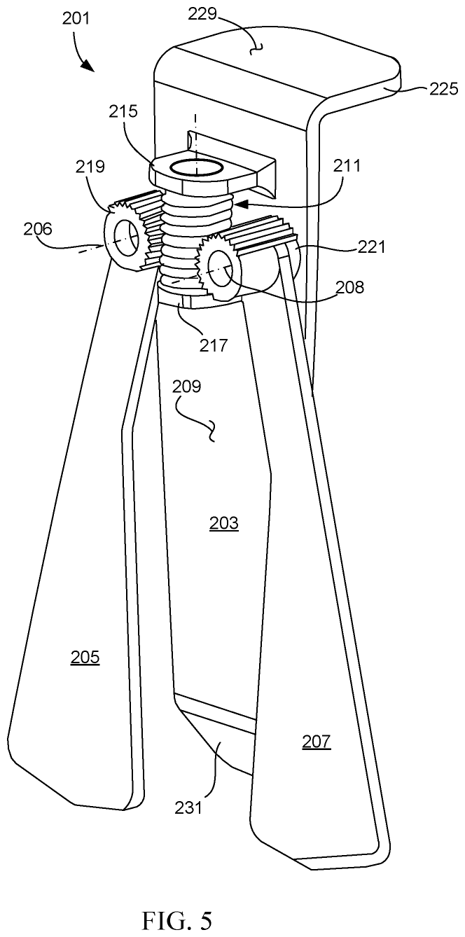

First blade 203 is configured to attach to a retractor tool commonly used in medical procedures, such as surgeries. One or more surfaces of blade 203 may be modified or aligned to facilitate attachment and function. As seen in the Figures, blade 203 may include an upper tab 225 which is angled from front surface 209. As shown it is orthogonal to front surface 209 but any angle may be used. Blade 203 may be bent over to form upper tab 225. This is similarly possible and applicable with assembly 101. An attachment device 227 can then releasably couple to a surface 229 of upper tab 225. Surface 229 can be any surface but is depicted as an upper surface for clarity purposes. Moreover, attachment device 227 is removed from FIGS. 5-9 for clarity purposes as well.

It should be known as well that blade 203 may further include a lower tab 231 also angled relative to front surface 209. The angle can be any desired. A purpose of lower tab 231 is to hold the tissue back and allows for increased visualization as a medical professional is looking downward from above. It assists in angling the tissue further rearward away from blades 205/207. Assembly 101 may have a similar lower tab as seen with assembly 201.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

1. An expandable blade assembly, comprising:
a first blade;
a first pivoting blade pivotal about a first axis, the first pivoting blade coupled to the first blade;
a second pivoting blade pivotal about a second axis and coupled to the first blade;
wherein the first pivoting blade and the second pivoting blade operate between a closed position and an open position, a collective width of the first blade, the first pivoting blade, and the second pivoting blade increases when transitioning from the closed position to the open position wherein the first blade extends between the first pivoting blade and the second pivoting blade when out of the closed position.

2. The assembly of claim 1, wherein the first pivoting blade and the second pivoting blade each pivot along a single unique plane.

3. The assembly of claim 1, wherein the first pivoting blade pivots about the first axis independent from that of the second pivoting blade about the second axis.

4. The assembly of claim 1, wherein first pivoting blade and the second pivoting blade pivot simultaneously about each respective axis.

5. The assembly of claim 4, wherein the pivoting of each blade is permitted in opposing directions.

6. The assembly of claim 1, wherein the first blade comprises a rear surface and is curved so as to extend outward from a rear surface.

7. An expandable blade assembly, comprising:
a first blade having a front surface and a rear surface;
a first pivoting blade pivotal about a first axis, the first pivoting blade coupled to the first blade;
a second pivoting blade pivotal about a second axis and coupled to the first blade; and
a gear coupled to the front surface of the first blade, the gear being in communication with the first pivoting blade and the second pivoting blade, the gear configured to rotate about a gear axis, the gear axis being misaligned with the first axis and the second axis;
wherein the first pivoting blade and the second pivoting blade operate between a closed position and an open position, a collective width of the first blade, the first pivoting blade, and the second pivoting blade increases when transitioning from the closed position to the open position wherein the first blade extends between the first pivoting blade and the second pivoting blade when out of the closed position; and
wherein rotation of the gear induces pivoting of the first pivoting blade and the second pivoting blade between the closed position and the open position.

8. The assembly of claim 7, wherein the first pivoting blade and the second pivoting blade each pivot along a single unique plane.

9. The assembly of claim 7, wherein the first pivoting blade pivots about the first axis independent from that of the second pivoting blade about the second axis.

10. The assembly of claim 7, wherein first pivoting blade and the second pivoting blade pivot simultaneously about each respective axis.

11. The assembly of claim 10, wherein the pivoting of each blade is permitted in opposing directions.

12. The assembly of claim 7, wherein the first blade is curved so as to extend outward from the rear surface.

13. The assembly of claim 7, wherein the gear axis is orthogonal to the first axis.

14. The assembly of claim 7, wherein the first pivoting blade includes a series of teeth configured to engage the gear.

15. The assembly of claim 7, wherein the first pivoting blade includes an engagement member at a first end of the first pivoting blade, the engagement member includes a series of teeth configured to engage the gear.

16. The assembly of claim 15, wherein the engagement member defines the first axis, the first pivoting blade extending out from an outer surface of the engagement member.

17. The assembly of claim 15, wherein the series of teeth are located along an outer surface of the engagement member.

18. The assembly of claim 7, further comprising:
an activator tool configured to seat within a portion of the gear to facilitate rotation of the gear.

19. A blade for use in an expandable blade assembly, comprising:
a main body having a uniform thickness between a first surface and a second surface along its length, the second surface opposite to the first surface;
an engagement member coupled to a first end of the main body, the engagement member defining an axis normal to the first surface and extending beyond both the first surface and the second surface of the main body; and
a surface treatment on an outer surface of the engagement member configured to permit rotation of the engagement member along a plane parallel to the first surface, the surface treatment being one or more threaded grooves.

20. The blade of claim 19, wherein the surface treatment on the engagement member extends around less than a full perimeter of the engagement member.
